SEQUENCE LISTING (4 sequences)

SEQ ID NO: 1 - protein, 1036 aa, synthetic construct
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 2 - protein, 894 aa, synthetic construct
YVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 3 - protein, 1291 aa, synthetic construct
MGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGYCAKYRAYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSWHHHHHH

SEQ ID NO: 4 - protein, 8 aa, synthetic construct
DYKDDDDK

(19) United States
(12) Patent Application Publication
(10) Pub. No.: US 2025/0155452 A1
(43) Pub. Date: May 15, 2025

(54) NLRP3 Variants & Library Screen for NLRP3 Inhibitors with the Same

(71) Applicant: BioAge Labs, Inc., Richmond, CA (US)

(72) Inventors: George H. Hartman, Lansdale, PA (US); Paul Humphries, Menlo Park, CA (US); Andrew Ho, Berkeley, CA (US); Robert Hughes, Berkeley, CA (US); Kevin Edward Leif Wilhelmsen, Richmond, CA (US); Rebecca Coll, Belfast (GB)

(21) Appl. No.: 18/906,535

(22) Filed: Oct. 4, 2024

Related U.S. Application Data
(60) Provisional application No. 63/587,779, filed on Oct. 4, 2023.

(51) Int. Cl. G01N 33/68; A61K 31/416; A61P 37/06; C07K 1/22; C07K 14/47; C07K 17/14; C12Q 1/6874; G01N 33/50

(52) U.S. Cl. CPC G01N 33/6869; A61K 31/416; A61P 37/06; C07K 1/22; C07K 14/4705; C07K 17/14; C12Q 1/6874; G01N 33/5023; G01N 33/5047; C07K 2319/21; C07K 2319/24; C12Q 2600/136; G01N 2500/10

(57) ABSTRACT

The present invention relates to a NLRP3 variant comprising at least a partial deletion of the PYD. The present invention further relates to a method of screening for a compound that inhibits the NLRP3 inflammasome. The present invention further provides a method comprising the step of administering a compound identified by such a screening method to a subject with a NLRP3-associated inflammatory disorder.

### CROSS REFERENCE TO RELATED APPLICATION

This application claims the benefit of U.S. Provisional Patent Application Ser. No. 63/587,779, filed Oct. 4, 2023. The entire content of the above-referenced patent application is incorporated by reference in its entirety herein.

### BACKGROUND

The NOD-like receptor (NLR) family, pyrin domain-containing protein 3 (NLRP3) inflammasome plays a central role in the innate immune response and inflammatory process.

Numerous diseases have been associated with aberrant NLRP3 activity. Modulation of the NLRP3 pathway via inhibition of NLRP3 may potentially provide an effective approach to alter the adverse clinical path of certain diseases. Numerous small molecule NLRP3 inhibitors have been identified and these compounds display a range of NLRP3 binding sites and reversible or irreversible mechanisms of action. Despite these advancements, there remains a need for new NLRP3 inhibitors and methods of identifying the same.

### SUMMARY

In one aspect, the disclosure provides a Nucleotide-binding oligomerization domain, Leucine rich Repeat and Pyrin domain containing 3 (NLRP3) variant, comprising at least a partial deletion of an amino terminal pyrin domain (PYD).

In certain embodiments, the NLRP3 variant comprises a deletion of amino acids 1-142 of the amino acid sequence of SEQ ID NO: 1.

In certain embodiments, the NLRP3 variant comprises an amino acid sequence with at least 80% identity to SEQ ID NO: 2.

In certain embodiments, the NLRP3 variant is linked to one or more polypeptide domains.

In certain embodiments, the one or more polypeptide domains comprise a solubility enhancing domain.

In certain embodiments, the one or more polypeptide domains are selected from the group consisting of maltose binding protein (MBP), glutathione S-transferase (GST), N-utilization substance A (NusA), and small ubiquitin-related modifier (SUMO).

In certain embodiments, the one or more polypeptide domains comprise an affinity purification domain.

In certain embodiments, the affinity purification domain comprises a His domain, 6×His domain, biotin, streptavidin, glutathione S-transferase (GST), FLAG (DYKDDDDK; SEQ ID NO: 4), and an antibody Fc domain.

In certain embodiments, the NLRP3 variant comprises an amino acid sequence with at least 80% identity to SEQ ID NO: 3.

In certain embodiments, the NLRP3 variant retains ATPase activity relative to a wild type NLRP3 of SEQ ID NO: 1.

In certain embodiments, the NLRP3 variant retains at least about 25% of ATPase activity relative to the ATPase activity of a wild type NLRP3 of SEQ ID NO: 1.

In certain embodiments, the NLRP3 variant retains the ability to form an NLRP3 inflammasome, relative to the ability of a wild type NLRP3 of SEQ ID NO: 1.

In certain embodiments, the NLRP3 variant retains the ability to initiate release of IL-1β in a cell, relative to the ability of a wild type NLRP3 of SEQ ID NO: 1.

In one aspect, the disclosure provides a nucleic acid encoding the NLRP3 variant described herein.

In one aspect, the disclosure provides a vector comprising the nucleic acid described herein.

In one aspect, the disclosure provides a host cell comprising the vector described herein.

In certain embodiments, the host cell is an E. coli cell, a yeast cell, an insect cell, or a mammalian cell.

In certain embodiments, the insect cell is an Sf21 cell.

In one aspect, the disclosure provides a method of purifying the NLRP3 variant described herein, comprising: 1) introducing a vector encoding the NLRP3 variant into a host cell; 2) culturing the host cell under conditions to allow expression of the NLRP3 variant in the host cell; and 3) isolating the NLRP3 variant from the host cell.

In certain embodiments, the isolating step 3) comprises contacting a lysate of the host cell with one or more affinity purification resins.

In certain embodiments, the one or more affinity purification resins are selected from the group consisting of an immobilized metal-affinity chromatography (IMAC) resin, a maltose or amylose affinity chromatography resin, a glutathione affinity chromatography resin, a protein A affinity chromatography resin, and an anti-FLAG affinity chromatography resin.

In one aspect, the disclosure provides a method of identifying a compound capable of binding to a Nucleotide-binding oligomerization domain, Leucine rich Repeat and Pyrin domain containing 3 (NLRP3) polypeptide, the method comprising: 1) contacting a compound suspected of being capable of binding an NLRP3 polypeptide with the NLRP3 variant described herein, wherein the NLRP3 variant is immobilized on a surface; 2) washing the compound and immobilized NLRP3 variant with a buffer; and 3) detecting the compound, wherein if the compound is detected, the compound is capable of binding an NLRP3 polypeptide.

In certain embodiments, the compound is linked to a polynucleotide sequence.

In certain embodiments, detection of the compound comprises detecting the polynucleotide sequence.

In certain embodiments, detecting the polynucleotide sequence comprises sequencing the polynucleotide sequence.

In certain embodiments, the polynucleotide sequence comprises at least 5 nucleotides.

In certain embodiments, the polynucleotide sequence is between 5 and 50 nucleotides in length.

In certain embodiments, the polynucleotide sequence is single stranded DNA (ssDNA).

In certain embodiments, the NLRP3 variant is at a concentration of between 1 nM and 300 nM.

In certain embodiments, the NLRP3 variant is at a concentration of between 25 nM and 250 nM.

In certain embodiments, the NLRP3 variant is contained in a buffer comprising: 1) 5 mM to 500 mM Tris-HCl; 2) 15 to 1500 mM NaCl; 3) 1 to 100 mM MgCl₂; 4) 1% to 20% Glycerol; and 5) 0.0005% to 0.05% Tween-20.

In certain embodiments, the buffer further comprises: 1) 1 to 500 mM imidazole; and 2) 0.03 to 3 mg/mL single-stranded DNA (ssDNA).

In certain embodiments, the NLRP3 variant is contained in a buffer comprising: 1) 50 mM Tris-HCl; 2) 150 mM NaCl; 3) 10 mM MgCl₂; 4) 10% Glycerol; and 5) 0.005% Tween-20.

In certain embodiments, the buffer further comprises: 1) 10 mM Imidazole; and 2) 0.3 mg/mL ssDNA.

In certain embodiments, the contacting step 1) comprises incubating the compound with the NLRP3 variant and between 0.01 mM to 50 mM ATP.

In certain embodiments, the contacting step 1) comprises incubating the compound with the NLRP3 variant and between 0.1 mM to 1 mM ATP.

In certain embodiments, the NLRP3 variant is immobilized on a solid support.

In certain embodiments, the solid support comprises a nickel-immobilized resin.

In certain embodiments, the method further comprises: 4) incubating the compound capable of binding an NLRP3 polypeptide with a cell; and 5) measuring at least one pro-inflammatory cytokine secreted from the cell, wherein a decrease in secretion of at least one pro-inflammatory cytokine from the cell indicates that the compound is an inhibitor of NLRP3.

In certain embodiments, the at least one pro-inflammatory cytokine is IL-1β.

In certain embodiments, the cell is a human monocytic THP-1 cell.

In one aspect, the disclosure provides a method of identifying an inhibitor of Nucleotide-binding oligomerization domain, Leucine rich Repeat and Pyrin domain containing 3 (NLRP3), the method comprising: 1) contacting a compound suspected of being capable of binding an NLRP3 polypeptide with the NLRP3 variant described herein, wherein the NLRP3 variant is immobilized on a surface; 2) washing the compound and immobilized NLRP3 variant with a buffer; 3) detecting the compound, wherein if the compound is detected, the compound is capable of binding an NLRP3 polypeptide; 4) incubating the compound capable of binding an NLRP3 polypeptide from step 3) with a cell; and 5) measuring at least one pro-inflammatory cytokine secreted from the cell, wherein a decrease in secretion of at least one pro-inflammatory cytokine from the cell indicates that the compound is an inhibitor of NLRP3.

In certain embodiments, the at least one pro-inflammatory cytokine is IL-1β.

In certain embodiments, the cell is a human monocytic THP-1 cell.

In one aspect, the disclosure provides a method of treating an inflammatory disorder, comprising administering to a subject the NLRP3 inhibitor identified herein.

In one aspect, the disclosure provides a composition comprising: 1) the NLRP3 variant described herein; 2) 5 mM to 500 mM Tris-HCl; 3) 15 to 1500 mM NaCl; 4) 1 to 100 mM MgCl₂; 5) 1% to 20% Glycerol; and 6) 0.0005% to 0.05% Tween-20.

In certain embodiments, the composition further comprises: 1) 1 to 500 mM imidazole; and 2) 0.03 to 3 mg/mL single-stranded DNA (ssDNA).

In certain embodiments, the composition comprises: 1) the NLRP3 variant described herein; 2) 50 mM Tris-HCl; 3) 150 mM NaCl; 4) 10 mM MgCl₂; 5) 10% Glycerol; and 6) 0.005% Tween-20.

In certain embodiments, the buffer further comprises: 1) 10 mM Imidazole; and 2) 0.3 mg/mL ssDNA.

In certain embodiments, the composition further comprises between 0.01 mM to 50 mM ATP.

### FIGURE DESCRIPTIONS

FIG. 1. Domain architecture of the MBP-ANLRP3-HIS protein used in the DEL screen.

FIG. 2. Outline for the construction of the key screening library.

FIG. 3. Generic structure for optimal ligands found in the DEL screen.

FIG. 4. Synthesis of analogs of compound 1 for NLRP3 inhibition evaluation in the THP-1. NLRP3 inflammasome activation assay.

FIG. 5. Luminescence of NLRP3 variant protein in the instant application buffer compared to literature buffer. Application buffer: 50 mM Tris-HCl, 150 mM NaCl, 10 mM MgCl₂, 10% Glycerol, 0.005% Tween-20, pH=7.5. Literature buffer: 20 mM Tris-HCl, 133 mM NaCl, 20 mM MgCl₂, 0.56 mM EDTA, 3 mM KCl, pH=7.8. Each buffer further comprised 10 μM ATP and a range of NLRP3 of 0 nM to 250 nM.

FIG. 6. Luminescence of NLRP3 variant protein in the instant application buffer with 0.3 mg/mL ssDNA, 10 mM imidazole, or both (selection buffer).

### DETAILED DESCRIPTION

Provided here is a method of identifying compounds such as peptides, inhibitors, and small molecules that can bind to Nucleotide-binding oligomerization domain, Leucine rich Repeat and Pyrin domain containing 3 (NLRP3). As used herein, the term “Nucleotide-binding oligomerization domain, Leucine rich Repeat and Pyrin domain containing 3” or “NOD-, LRR-, and pyrin domain-containing protein 3” or “NLRP3” refers to UNIPROT reference number Q96P20 and the amino acid sequence of SEQ ID NO: 1.

### NLRP3

NLRP3 is a member of the Nod-like receptor (NLR) family of proteins. NLRP3 is an intracellular sensor that detects a broad range of danger signals and environmental insults resulting in a protective pro-inflammatory response designed to impair pathogens and repair tissue damage via the formation and activation of the NLRP3 inflammasome (Coll, R. C., et al., Trends Pharmacol. Sci., 2022, 43(8), 653-668). NLRP3 is highly expressed in subsets of peripheral leukocytes and microglia of the central nervous system.

NLRP3 displays a tripartite structure consisting of a Pyrin Domain (PYD), a central nucleotide binding and oligomerization domain of the NACHT subfamily of NTPases, and a Leucine Rich Repeat domain (LRR). The NACHT domain has ATPase activity that is required to induce an inactive ADP-bound decameric assembly in cells (Hochheiser, I., et al., Nature, 2022, 604, 184-189), (Brinkschulte, R., et al., Commun. Biol., 2022, 5, 1176).

Assembly of the NLRP3 inflammasome leads to caspase 1-dependent secretory release of the pro-inflammatory cytokines IL-13 and IL-18 as well as to gasdermin D-mediated pyroptotic cell death.

### NLRP3 Variants

The disclosure relates to an NLRP3 variant useful for screening of NLRP3 inhibitors, such as the screening methods described herein.

In one aspect, the disclosure provides an NLRP3 variant comprising at least a partial deletion of an amino terminal pyrin domain (PYD).

As used herein, a “partial deletion” includes the deletion of at least one amino acid that reduces or eliminates the activity of the PYD, or maintains or increases stability of the protein as compared to the wild type protein of SEQ ID NO: 1.

As used herein, “wild type protein” includes the amino acid sequence of SEQ ID NO: 1 and naturally occurring variants or isoforms of NLRP3.

In certain embodiments, the partial deletion comprises a deletion of at least 1, 5, 10, 25, 50, 75, 100, 125, 142, 150, 175, 200, 216 amino acids of the PYD of NLRP3.

In certain embodiments, the NLRP3 variant comprises a deletion of amino acids 1-50, 1-90, 1-100, 1-142, 1-216, 3-110, 6-90, 7-12, and 8-108, from the N terminus of the amino acid sequence of SEQ ID NO: 1.

In certain embodiments, the NLRP3 variant comprises a deletion of amino acids 6-16, 17-20, 21-31, 32-42, 43-48, 49-50, 51-62, 62-63, 64-78, 79-80, and 81-90, from the N terminus of the amino acid sequence of SEQ ID NO: 1.

In certain embodiments, the NLRP3 variant comprises an amino acid sequence with at least 80% identity (e.g., 80%, 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 2.

In certain embodiments, the NLRP3 variant comprises an amino acid sequence with at least 80% identity (e.g., 80%, 81%, 82%, 83%, 84%, 85%, 86%, 87%, 88%, 89%, 90%, 91%, 92%, 93%, 94%, 95%, 96%, 97%, 98%, 99%, or 100% identity) to SEQ ID NO: 3.

In certain embodiments, the NLRP3 variant retains ATPase activity relative to a wild type NLRP3 of SEQ ID NO: 1. One of skill in the art can readily identify a method of measuring ATPase activity. For example, but in no way limiting, measuring the liberation of inorganic phosphate arising from the hydrolysis of ATP by colorimetric methods. For example, but in no way limiting, ATPase activity can be measured by ADP formed from a kinase reaction by converting the ADP into ATP, which is used to generate light in a luciferase reaction. The luminescence generated correlates with kinase activity.

In certain embodiments, the NLRP3 variant retains at least about 25% of ATPase activity relative to the ATPase activity of a wild type NLRP3 of SEQ ID NO: 1. In certain embodiments, the NLRP3 variant has more than 100% of ATPase activity relative to the ATPase activity of a wild type NLRP3 of SEQ ID NO: 1.

In certain embodiments, the NLRP3 variant retains at least about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of ATPase activity relative to the ATPase activity of a wild type NLRP3 of SEQ ID NO: 1.

In certain embodiments, the NLRP3 variant retains the ability to form an NLRP3 inflammasome, relative to the ability of a wild type NLRP3 of SEQ ID NO: 1.

In certain embodiments, the NLRP3 variant retains the ability to initiate release of IL-1P in a cell, relative to the ability of a wild type NLRP3 of SEQ ID NO: 1.

The instant disclosure also provides nucleic acids encoding the NLRP3 variants disclosed herein. In certain embodiments, the disclosure provides a vector comprising the nucleic acid sequence encoding the NLRP3 variant. In certain embodiments, the disclosure provides a host cell comprising the vector encoding the NLRP3 variant. In certain embodiments, said host cell is an E. coli cell, a yeast cell, an insect cell, or a mammalian cell. In certain embodiments, the host cell an insect Sf21 cell.

### Purification of NLRP3 Variants

The present invention features a method for purifying NLRP3 variants disclosed herein.

In one aspect, the disclosure provides a method of purifying the NLRP3 variant described herein, the method comprising: 1) introducing a vector encoding the NLRP3 variant into a host cell; 2) culturing the host cell under conditions to allow expression of the NLRP3 variant in the host cell; and 3) isolating the NLRP3 variant from the host cell.

In certain embodiments, the isolating step 3) comprises contacting a lysate of the host cell with one or more affinity purification resins. In other embodiments, the NLPR3 variant is secreted into the host cell culture media. In such embodiments, the host cell culture media containing the NLRP3 variant is contacted with one or more affinity purification resins.

In certain embodiments, the one or more affinity purification resins are selected from the group consisting of an immobilized metal-affinity chromatography (IMAC) resin, a maltose or amylose affinity chromatography resin, a glutathione affinity chromatography resin, a protein A affinity chromatography resin, and a anti-FLAG affinity chromatography resin.

In certain embodiments, the host cell used for expression of the NLRP3 variant for purification is an E. coli cell, a yeast cell, an insect cell, or a mammalian cell. In certain embodiments, the insect cell is an Sf21 cell.

### NLRP3 Variant Fusions

The NLRP3 variants may be linked to one or more non-NLRP3 polypeptide domains. The additional polypeptide domains confer a non-natural activity (i.e., an activity not found in WT NLRP3) upon the NLRP3 variant.

In certain embodiments, the one or more polypeptide domains comprise a solubility enhancing domain. As used herein, the term “solubility enhancing domain” refers to a polypeptide that enhances the solubility of a protein to which is it attached.

In certain embodiments, the one or more polypeptide domains (e.g., the solubility enhancing domain) are selected from the group consisting of maltose binding protein (MBP), glutathione S-transferase (GST), N-utilization substance A (NusA), and small ubiquitin-related modifier (SUMO). Solubility enhancing domains are described in further detail in Berneir et al. Protein Expr Purif. 2018. 152:92-106.

In certain embodiments, the one or more polypeptide domains comprise an affinity purification domain.

In certain embodiments, the affinity purification domain comprises a His domain, 6×His domain, biotin, streptavidin, glutathione S-transferase (GST), FLAG (DYKDDDDK; SEQ ID NO: 4), and an antibody Fc domain.

### Identification of Compounds Capable of Binding NLRP3

The NLRP3 variants of the disclosure are useful for the identification of compounds capable of binding to NLRP3. Said binding compounds may be further tested to determine if the compounds are capable of inhibiting NLRP3. For example, but in no way limiting, inhibition of NLRP3 can be reflected in the ability to form the NLRP3 inflammasome or initiate release of IL-1β. Inhibiting NLRP3 inflammasome activity may be accomplished using any method known to the skilled artisan.

As used herein, the term “DNA encoded chemical library” or “DEL” refers to a mixture of small molecules conjugated to unique polynucleotide sequence tags, with structural information of each small molecule encoded into its corresponding sequence. See (Brenner, S., Lerner, R. A., Proc. Natl. Acad. Sci., 1992, 89, 5381-5383), (Gartner, Z. J. Science, 2004, 305, 1601-1605), (Buller F., et al, Bioconjug. Chem., 2010, 21, 1571-1580), incorporated herein by reference. When DELs are applied against proteins of interest in selection conditions, library compounds with highest affinity are enriched after multiple washes, and DNA sequencing is used to elucidate their structural identity (Clark M. A., et al., Nat. Chem. Biol., 2009, 5, 647-654).

In certain embodiments, the NLRP3 variant described herein is contacted with a DNA encoded chemical library (DEL) in a buffer. In certain embodiments, the NLRP3 variant DEL library composition is applied to a solid support, such as Ni-charged magnetic beads. In certain embodiments, the Ni-charged beads with bound NLRP3 variant and DEL library are washed one or more times, and subsequently the NLRP3 variant and DNA encoded library members (DELs) are released into the supernatant (Chen, Q., et al, SLAS Discov., 2020, 25(5), 523-529).

In certain embodiments, the resulting DEL library composition is subjected to identification. In certain embodiments, identification is ascertained following PCR amplification and DNA sequencing.

Thus, in one aspect, the disclosure provides a method of identifying a compound capable of binding to a Nucleotide-binding oligomerization domain, Leucine rich Repeat and Pyrin domain containing 3 (NLRP3) polypeptide, the method comprising: 1) contacting a compound suspected of being capable of binding an NLRP3 polypeptide with the NLRP3 variant described herein, wherein the NLRP3 variant is immobilized on a surface; 2) washing the compound and immobilized NLRP3 variant with a buffer; and 3) detecting the compound, wherein if the compound is detected, the compound is capable of binding an NLRP3 polypeptide.

As used herein, the term “compound suspected of being capable of binding an NLRP3 polypeptide” refers to a test compound used in the methods of identifying a compound described herein. In certain embodiments, the test compound comprises an indazole. In certain embodiments, the test compound comprises an indazole linked to a biphenyl subunit.

In certain embodiments, the test compound comprises an indazole functionalized at C-7 via a 3-atom carbonylaminomethyl linker connected to a biphenyl subunit.

In certain embodiments, the compound is linked to a polynucleotide sequence.

In certain embodiments, detection of the compound comprises detecting the polynucleotide sequence.

In certain embodiments, detecting the polynucleotide sequence comprises sequencing the polynucleotide sequence.

In certain embodiments, the polynucleotide sequence comprises at least 5 nucleotides.

In certain embodiments, the polynucleotide sequence is between 5 and 100 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length). In certain embodiments, the polynucleotide sequence is between 5 and 50 nucleotides in length.

In certain embodiments, the polynucleotide sequence is single stranded DNA (ssDNA).

In certain embodiments, a plurality of compounds suspected of being capable of binding an NLRP3 polypeptide are contacted with the NLRP3 variants described herein, each compound of the plurality is linked to a unique polynucleotide sequence. Thus, each compound in the plurality can be identified by sequencing the unique polynucleotide sequence.

In certain embodiments, the NLRP3 variant is at a concentration of between 1 nM and 300 nM (e.g., 1 nM, 5 nM, 10 nM, 15 nM, 20 nM, 25 nM, 50 nM, 75 nM, 100 nM, 150 nM, 200 nM, 250 nM, or 300 nM). In certain embodiments, the NLRP3 variant is at a concentration of between 25 nM and 250 nM.

In certain embodiments, the NLRP3 variant is contained in a buffer comprising: 1) 5 mM to 500 mM Tris-HCl (e.g., 5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM, or 500 mM); 2) 15 to 1500 mM NaCl (e.g., 15 mM, 50 mM, 100 mM 125 mM, 140 mM, 145 mM, 150 mM, 155 mM, 160 mM, 165 mM, 170 mM, 175 mM, 180 mM, 185 mM, 190 mM, 195 mM, 200 mM, 300 mM, 400 mM, 500 mM, 1,000 mM, or 1,500 mM); 3) 1 to 100 mM MgCl₂ (e.g., 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 50 mM, 75 mM, or 100 mM); 4) 1% to 20% Glycerol (e.g., 1%, 2%, 3%, 4%, 5%, 6%, 7%, 8%, 9%, 10%, 11%, 12%, 13%, 14%, 15%, 16%, 17%, 18%, 19%, or 20%); and 5) 0.0005% to 0.05% Tween-20 (e.g., 0.0005%, 0.001%, 0.005%, 0.01%, or 0.05%).

In certain embodiments, the buffer further comprises 1 to 500 mM imidazole (e.g., 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM).

In certain embodiments, the buffer further comprises 0.03 to 3 mg/mL single-stranded DNA (ssDNA) (e.g., 0.03, 0.1, 0.5, 1.0, 1.5, 2.0, 2.5, or 3.0 mg/mL).

In certain embodiments, the NLRP3 variant is contained in a buffer comprising: 1) 25-75 mM Tris-HCl; 2) 140-160 mM NaCl; 3) 5-15 mM MgCl₂; 4) 5%-10% Glycerol; and 5) 0.001%-0.01% Tween-20.

In certain embodiments, the NLRP3 variant is contained in a buffer comprising: 1) 50 mM Tris-HCl; 2) 150 mM NaCl; 3) 10 mM MgCl₂; 4) 10% Glycerol; and 5) 0.005% Tween-20.

In certain embodiments, the buffer further comprises: 1) 10 mM Imidazole; and 2) 0.3 mg/mL ssDNA.

In certain embodiments, the contacting step 1) comprises incubating the compound with the NLRP3 variant and between 0.01 mM to 50 mM ATP.

In certain embodiments, the contacting step 1) comprises incubating the compound with the NLRP3 variant and between 0.1 mM to 1 mM ATP.

In certain embodiments, the NLRP3 variant is immobilized on a solid support.

In certain embodiments, the solid support is conjugated to a ligand that binds the NLRP3 variant. In certain embodiments, the solid support comprises a nickel-immobilized resin. In certain embodiments, the solid support comprises immobilized metal-affinity (IMAC) resin, a maltose or amylose affinity resin, a glutathione affinity resin, a protein A affinity resin, or an anti-FLAG affinity resin.

In certain embodiments, the solid support comprises a nickel-immobilized resin.

In certain embodiments, the method further comprises: 4) incubating the compound capable of binding an NLRP3 polypeptide with a cell; and 5) measuring at least one pro-inflammatory cytokine secreted from the cell, wherein a decrease in secretion of at least one pro-inflammatory cytokine from the cell indicates that the compound is an inhibitor of NLRP3.

In certain embodiments, the at least one pro-inflammatory cytokine is IL-1β.

In certain embodiments, the cell is a human monocytic THP-1 cell.

In certain embodiments, the compound identified as an NLRP3 inhibitor is capable of decreasing IL-1P secretion from a cell contacted with said compound identified as an NLRP3 inhibitor. In certain embodiments, the cell is a human monocytic THP-1 cell.

Identification of inhibitors of NLRP3

In one aspect, the disclosure provides a method of identifying an inhibitor of Nucleotide-binding oligomerization domain, Leucine rich Repeat and Pyrin domain containing 3 (NLRP3), the method comprising: 1) contacting a compound suspected of being capable of binding an NLRP3 polypeptide with the NLRP3 variant described herein, wherein the NLRP3 variant is immobilized on a surface; 2) washing the compound and immobilized NLRP3 variant with a buffer; 3) detecting the compound, wherein if the compound is detected, the compound is capable of binding an NLRP3 polypeptide; 4) incubating the compound capable of binding an NLRP3 polypeptide from step 3) with a cell; and 5) measuring at least one pro-inflammatory cytokine secreted from the cell, wherein a decrease in secretion of at least one pro-inflammatory cytokine from the cell indicates that the compound is an inhibitor of NLRP3.

In certain embodiments, the compound is linked to a polynucleotide sequence.

In certain embodiments, detection of the compound comprises detecting the polynucleotide sequence.

In certain embodiments, detecting the polynucleotide sequence comprises sequencing the polynucleotide sequence.

In certain embodiments, the polynucleotide sequence comprises at least 5 nucleotides.

In certain embodiments, the polynucleotide sequence is between 5 and 100 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length). In certain embodiments, the polynucleotide sequence is between 5 and 50 nucleotides in length.

In certain embodiments, the polynucleotide sequence is single stranded DNA (ssDNA).

In certain embodiments, a plurality of compounds suspected of being capable of binding an NLRP3 polypeptide are contacted with the NLRP3 variants described herein, each compound of the plurality is linked to a unique polynucleotide sequence. Thus, each compound in the plurality can be identified by sequencing the unique polynucleotide sequence.

In certain embodiments, the NLRP3 variant is at a concentration of between 1 nM and 300 nM (e.g., 1 nM, 5 nM, 10 nM, 15 nM, 20 nM, 25 nM, 50 nM, 75 nM, 100 nM, 150 nM, 200 nM, 250 nM, or 300 nM). In certain embodiments, the NLRP3 variant is at a concentration of between 25 nM and 250 nM.

In certain embodiments, the NLRP3 variant is contained in a buffer comprising: 1) 5 mM to 500 mM Tris-HCl (e.g., 5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM, or 500 mM); 2) 15 to 1500 mM NaCl (e.g., 15 mM, 50 mM, 100 mM 125 mM, 140 mM, 145 mM, 150 mM, 155 mM, 160 mM, 165 mM, 170 mM, 175 mM, 180 mM, 185 mM, 190 mM, 195 mM, 200 mM, 300 mM, 400 mM, 500 mM, 1,000 mM, or 1,500 mM); 3) 1 to 100 mM MgCl₂ (e.g., 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 50 mM, 75 mM, or 100 mM); 4) 1% to 20% Glycerol (e.g., 1%, 2%, 3%, 4%, 5%, 6%, 7%, 8%, 9%, 10%, 11%, 12%, 13%, 14%, 15%, 16%, 17%, 18%, 19%, or 20%); and 5) 0.0005% to 0.05% Tween-20 (e.g., 0.0005%, 0.001%, 0.005%, 0.01%, or 0.05%).

In certain embodiments, the buffer further comprises 1 to 500 mM imidazole (e.g., 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM).

In certain embodiments, the buffer further comprises 0.03 to 3 mg/mL single-stranded DNA (ssDNA) (e.g., 0.03, 0.1, 0.5, 1.0, 1.5, 2.0, 2.5, or 3.0 mg/mL).

In certain embodiments, the NLRP3 variant is contained in a buffer comprising: 1) 25-75 mM Tris-HCl; 2) 140-160 mM NaCl; 3) 5-15 mM MgCl₂; 4) 5%-10% Glycerol; and 5) 0.001%-0.01% Tween-20.

In certain embodiments, the NLRP3 variant is contained in a buffer comprising: 1) 50 mM Tris-HCl; 2) 150 mM NaCl; 3) 10 mM MgCl₂; 4) 10% Glycerol; and 5) 0.005% Tween-20.

In certain embodiments, the buffer further comprises: 1) 10 mM Imidazole; and 2) 0.3 mg/mL ssDNA.

In certain embodiments, the contacting step 1) comprises incubating the compound with the NLRP3 variant and between 0.01 mM to 50 mM ATP.

In certain embodiments, the contacting step 1) comprises incubating the compound with the NLRP3 variant and between 0.1 mM to 1 mM ATP.

In certain embodiments, the NLRP3 variant is immobilized on a solid support.

In certain embodiments, the solid support is conjugated to a ligand that binds the NLRP3 variant. In certain embodiments, the solid support comprises a nickel-immobilized resin. In certain embodiments, the solid support comprises immobilized metal-affinity (IMAC) resin, a maltose or amylose affinity resin, a glutathione affinity resin, a protein A affinity resin, or an anti-FLAG affinity resin.

In certain embodiments, the solid support comprises a nickel-immobilized resin.

In certain embodiments, the at least one pro-inflammatory cytokine is IL-1β.

In certain embodiments, the cell is a human monocytic THP-1 cell.

### NLRP3 Variant Buffer

In application provides NLRP3 variant buffer compositions that enhance stability of the NLRP3 variants.

In certain embodiments, the NLRP3 variant is contained in a buffer comprising: 1) 5 mM to 500 mM Tris-HCl (e.g., 5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM, or 500 mM); 2) 15 to 1500 mM NaCl (e.g., 15 mM, 50 mM, 100 mM 125 mM, 140 mM, 145 mM, 150 mM, 155 mM, 160 mM, 165 mM, 170 mM, 175 mM, 180 mM, 185 mM, 190 mM, 195 mM, 200 mM, 300 mM, 400 mM, 500 mM, 1,000 mM, or 1,500 mM); 3) 1 to 100 mM MgCl₂ (e.g., 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 50 mM, 75 mM, or 100 mM); 4) 1% to 20% Glycerol (e.g., 1%, 2%, 3%, 4%, 5%, 6%, 7%, 8%, 9%, 10%, 11%, 12%, 13%, 14%, 15%, 16%, 17%, 18%, 19%, or 20%); and 5) 0.0005% to 0.05% Tween-20 (e.g., 0.0005%, 0.001%, 0.005%, 0.01%, or 0.05%).

In certain embodiments, the buffer further comprises 1 to 500 mM imidazole (e.g., 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM).

In certain embodiments, the buffer further comprises 0.03 to 3 mg/mL single-stranded DNA (ssDNA) (e.g., 0.03, 0.1, 0.5, 1.0, 1.5, 2.0, 2.5, or 3.0 mg/mL).

In certain embodiments, the NLRP3 variant is contained in a buffer comprising: 1) 25-75 mM Tris-HCl; 2) 140-160 mM NaCl; 3) 5-15 mM MgCl₂; 4) 5%-10% Glycerol; and 5) 0.001%-0.01% Tween-20.

In certain embodiments, the NLRP3 variant is contained in a buffer comprising: 1) 50 mM Tris-HCl; 2) 150 mM NaCl; 3) 10 mM MgCl₂; 4) 10% Glycerol; and 5) 0.005% Tween-20.

In certain embodiments, the buffer further comprises: 1) 10 mM Imidazole; and 2) 0.3 mg/mL ssDNA.

### Methods of Treatment

In an aspect, provided herein is a method of inhibiting NLRP3 inflammasome in a subject in need thereof, comprising administering to the individual a therapeutically effective amount of a compound disclosed herein (i.e., an NLRP3 inhibitor identified in the screening methods disclosed herein).

In another aspect, provided herein is a method of treating inflammation in a subject in need thereof, comprising administering to the individual a therapeutically effective amount of a compound disclosed herein.

In another aspect, provided herein is a method of treating inflammation in a subject in need thereof, comprising administering to the individual a therapeutically effective amount of a compound disclosed herein.

In another aspect, provided herein is a method of treating cryopyrin-associated periodic syndrome (CAPS) in a subject in need thereof, comprising administering to the individual a therapeutically effective amount of a compound disclosed herein.

In an embodiment, the CAPS is selected from the group consisting of familial cold autoinflammatory syndrome, Muckle-Wells syndrome, and neonatal-onset multisystem inflammatory disease.

In another aspect, provided herein is a method of treating a dermatologic disease in a subject in need thereof, comprising administering to the individual a therapeutically effective amount of a compound disclosed herein.

In an embodiment, the dermatologic disease is selected from the group consisting of psoriasis, urticaria, skin photoaging, and eczema.

In yet another aspect, provided herein is a method of treating a neurosensory disease in a subject in need thereof, comprising administering to the individual a therapeutically effective amount of a compound disclosed herein.

In an embodiment, the neurosensory disease is selected from the group consisting of amyotrophic lateral sclerosis (ALS), traumatic brain injury, Parkinson's disease, and Alzheimer's disease.

Also provided herein is a method of using the compounds provided herein for treatment or amelioration of aging or an aging-related condition negatively impacting longevity or quality of life, wherein the aging-related condition negatively impacting longevity or quality of life is selected from the group consisting of inflammation, anemia, hyperglycemia, dyslipidemia, hyperinsulinemia, insulin resistance, immunosuppression, liver disease, iron overload, hypertrigliceridemia, impaired skin integrity, wound healing, scarring, pain, allergies, sleep disorders and problems, gastrointestinal disorders and problems, Th1-type inflammation, Th2-type inflammation, an inflammatory disease involving T-cell dependent B cell proliferation, T-cell dependent B cell proliferation, allergy, asthma, atherosclerosis, autoimmunity, hypercholesterolemia, chronic inflammation, chronic obstructive pulmonary disease (COPD), Crohn's disease, cutaneous responses to tissue damage, fibrosis, hematological oncology, metabolic diseases, cardiovascular disease, organ transplantation, psoriasis, liver fibrosis, dermatitis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and diseases that are driven or exacerbated by one or more factors selected from the group consisting of alpha smooth muscle actin (aSMA), CD40, CD69, collagen I, collagen Ill, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1.alpha., IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAl-1), prostaglandin E2 (PGE2), serum amyloid A, T or B cell proliferation, tissue plasminogen activator (tPA), tumor necrosis factor alpha (TNF.alpha.), vascular cell adhesion molecule (VCAM-1), and vascular endothelial growth factor 2 (VEGFR2), comprising: administering to a subject in need thereof a compound provided herein.

In an aspect, provided herein is a method of reversing a normal aging process in subject comprising administering to the subject a therapeutically effective amount of a compound provided herein or a pharmaceutically acceptable salt thereof.

In another aspect, provided herein is a method of reversing a normal aging process in subject comprising administering to the subject a pharmaceutical composition comprising a therapeutically effective amount of a compound provided herein or a pharmaceutically acceptable salt thereof.

In yet another aspect, provided herein is a method of extending lifespan of a subject comprising administering to the subject a therapeutically effective amount of a compound provided herein or a pharmaceutically acceptable salt thereof.

In still another aspect, provided herein is a method of extending lifespan of a subject comprising administering to the subject a pharmaceutical composition comprising a therapeutically effective amount of a compound provided herein or a pharmaceutically acceptable salt thereof.

In another aspect, provided herein is a method to slow down and mitigate the aging process in a subject comprising administering to the subject a therapeutically effective amount of a compound provided herein or a pharmaceutically acceptable salt thereof.

In another aspect, provided herein is a method of inhibiting or modulating the pro-inflammatory pathway in a cell comprising contacting the cell with a compound provided herein, or a pharmaceutically acceptable salt thereof. In yet another aspect, provided herein is a method of inhibiting or modulating NLRP3 in a cell comprising contacting the cell with a compound provided herein, or a pharmaceutically acceptable salt thereof.

Treatment of a cell (in vitro or in vivo) that expresses a NLRP3 inflammasome with a compound provided herein can result in inhibiting the pro-inflammatory pathway and inhibiting downstream events related to the signaling pathway such as inflammation or inflammaging.

In another aspect, provided herein is a method of treating a neurosensory disease in a subject in need thereof, comprising administering to the individual a therapeutically effective amount of a compound disclosed herein.

In an embodiment, the neurosensory disease is selected from the group consisting of hearing loss, hearing injury, and ocular disease. In an embodiment, the ocular disease is retinal and optic nerve injury.

In yet another aspect, provided herein is a method of treating an inflammatory disorder in a subject in need thereof, comprising administering to the individual a therapeutically effective amount of a compound disclosed herein.

In an embodiment, the inflammatory disorder is selected from the group consisting of allergy, asthma, atopic dermatitis, atherosclerosis, autoimmune diseases, coeliac disease, chronic inflammation, glomerulonephritis, hepatitis, inflammatory bowel disease, preperfusion injury, SARS-CoV-2 infection, transplant rejection, heart disease, diabetes, arthritis, Crohn's disease, ulcerative colitis, non-alcoholic steatohepatitis (NASH), gout, coronary artery disease, rheumatoid arthritis, intestinal disorders, and acute respiratory distress syndrome (ARDS).

In another embodiment, the inflammatory disorder is a neuroinflammatory disease. In yet another embodiment, the inflammatory disorder is inner ear inflammation.

In an embodiment, a chronic inflammation comprises a tissue inflammation. Tissue inflammation is a chronic inflammation that is confined to a particular tissue or organ. In an embodiment, a tissue inflammation comprises, e.g., a skin inflammation, ocular inflammation, a muscle inflammation, a tendon inflammation, a ligament inflammation, a bone inflammation, a cartilage inflammation, a lung inflammation, a heart inflammation, a liver inflammation, a pancreatic inflammation, a kidney inflammation, a bladder inflammation, a stomach inflammation, an intestinal inflammation, a neuron inflammation, and a brain inflammation.

In another embodiment, a chronic inflammation comprises a systemic inflammation. Although the processes involved are identical to tissue inflammation, systemic inflammation is not confined to a particular tissue but in fact overwhelms the body, involving the endothelium and other organ systems. When it is due to infection, the term sepsis is applied, with the terms bacteremia being applied specifically for bacterial sepsis and viremia specifically to viral sepsis. Vasodilation and organ dysfunction are serious problems associated with widespread infection that may lead to septic shock and death.

In yet another embodiment, a chronic inflammation comprises an arthritis. Arthritis includes a group of conditions involving damage to the joints of the body due to the inflammation of the synovium including, without limitation osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

In still another aspect, provided herein is a method of treating an age-related disorder in a subject in need thereof, comprising administering to the individual a therapeutically effective amount of a compound disclosed herein.

In an embodiment, the age-related disorder is selected from the group consisting of neurodegeneration, cardiovascular disease, insulin resistance, diabetes, osteoporosis, osteoarthritis, cognitive decline, dementia, frailty, cataracts, arthritis, obesity, hypertension, angina, congestive heart failure, dyslipidemia, myocardial infarction, vascular disease, respiratory disease, kidney disease, cerebrovascular disease, peripheral vascular disease, Alzheimer's disease, cardiac diastolic dysfunction, benign prostatic hypertrophy, aortic aneurysm, and emphysema.

In another aspect, provided herein is a method of treating a metabolic condition in a subject in need thereof, comprising administering to the individual a therapeutically effective amount of a compound disclosed herein.

In an embodiment, the metabolic condition is selected from the group consisting of diabetes, obesity, cystic fibrosis, and hyperthyroidism.

In yet another aspect, provided herein is a method of treating a neurodegenerative disease in a subject in need thereof, comprising administering to the individual a therapeutically effective amount of a compound disclosed herein.

In an embodiment, the neurodegenerative disease is selected from the group consisting of Alzheimer's disease, Parkinson's disease, Huntington's disease, multiple sclerosis (MS), amyotrophic lateral sclerosis (ALS), and Batten disease.

In an aspect, provided herein is a method of treating a disease or disorder of the inner ear in a subject in need thereof, comprising administering to the individual a therapeutically effective amount of a compound disclosed herein.

In an embodiment, the disease or disorder of the inner ear is selected from the group consisting of hearing loss, hearing impairment, vertigo, Meniere's disease, and tinnitus. In another embodiment, the disease of the inner ear is hearing loss. In yet another embodiment, the disease of the inner ear is hearing impairment.

In another embodiment, the hearing loss is age-related, noise-induced, or the result of a viral infection. In yet another embodiment, the viral infection is Zika virus or coronavirus.

Potency of the inhibitor can also be determined by IC₅₀ value. A compound with a lower IC₅₀ value, as determined under substantially similar conditions, is a more potent inhibitor relative to a compound with a higher IC₅₀ value.

In an embodiment of the methods, the subject is a human.

In another aspect, the disclosure provides a compound disclosed herein, or a pharmaceutically acceptable salt thereof, for use in the manufacture of a medicament for treating or preventing a disease in which NLRP3 inflammasome plays a role.

In an aspect, provided herein is a method of treating a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer.

Therefore, in an aspect, provided herein is a method of treating cancer in a subject in need thereof comprising administering to the subject a therapeutically effective amount of a compound disclosed herein, or a pharmaceutically acceptable salt thereof.

In an embodiment, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In another embodiment, the cancer is selected from the group consisting of myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

In an embodiment, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In an embodiment, the cancer is selected from the group consisting of tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In another aspect, provided herein is the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

Sequence ID Numbers

| SEQ ID | | |
|---|---|---|
| NO | Construct | Amino Acid Sequence |
| SEQ ID | NLRP3 | MKMASTRCKLARYLEDLEDVDLKKFKMHLEDYPPQKGCIPLPRGQTEKADHV |
| NO 1 | | DLATLMIDFNGEEKAWAMAVWIFAAINRRDLYEKAKRDEPKWGSDNARVSNP |
| | | TVICQEDSIEEEWMGLLEYLSRISICKMKKDYRKKYRKYVRSRFQCIEDRNARL |
| | | GESVSLNKRYTRLRLIKEHRSQQEREQELLAIGKTKTCESPVSPIKMELLFDPD |
| | | DEHSEPVHTVVFQGAAGIGKTILARKMMLDWASGTLYQDRFDYLFYIHCREVS |
| | | LVTQRSLGDLIMSCCPDPNPPIHKIVRKPSRILFLMDGFDELQGAFDEHIGPLC |
| | | TDWQKAERGDILLSSLIRKKLLPEASLLITTRPVALEKLQHLLDHPRHVEILGFS |
| | | EAKRKEYFFKYFSDEAQARAAFSLIQENEVLFTMCFIPLVCWIVCTGLKQQME |
| | | SGKSLAQTSKTTTAVYVFFLSSLLQPRGGSQEHGLCAHLWGLCSLAADGIWN |
| | | QKILFEESDLRNHGLQKADVSAFLRMNLFQKEVDCEKFYSFIHMTFQEFFAAM |
| | | YYLLEEEKEGRTNVPGSRLKLPSRDVTVLLENYGKFEKGYLIFVVRFLFGLVNQ |
| | | ERTSYLEKKLSCKISQQIRLELLKWIEVKAKAKKLQIQPSQLELFYCLYEMQEED |
| | | FVQRAMDYFPKIEINLSTRMDHMVSSFCIENCHRVESLSLGFLHNMPKEEEEE |
| | | EKEGRHLDMVQCVLPSSSHAACSHGLVNSHLTSSFCRGLFSVLSTSQSLTEL |
| | | DLSDNSLGDPGMRVLCETLQHPGCNIRRLWLGRCGLSHECCFDISLVLSSNQ |
| | | KLVELDLSDNALGDFGIRLLCVGLKHLLCNLKKLWLVSCCLTSACCQDLASVLS |
| | | TSHSLTRLYVGENALGDSGVAILCEKAKNPQCNLQKLGLVNSGLTSVCCSALS |
| | | SVLSTNQNLTHLYLRGNTLGDKGIKLLCEGLLHPDCKLQVLELDNCNLTSHCC |
| | | WDLSTLLTSSQSLRKLSLGNNDLGDLGVMMFCEVLKQQSCLLQNLGLSEMYF |
| | | NYETKSALETLQEEKPELTVVFEPSW |
| SEQ ID | ΔNLRP3 | YVRSRFQCIEDRNARLGESVSLNKRYTRLRLIKEHRSQQEREQELLAIGKTKT |
| NO 2 | | CESPVSPIKMELLFDPDDEHSEPVHTVVFQGAAGIGKTILARKMMLDWASGTL |
| | | YQDRFDYLFYIHCREVSLVTQRSLGDLIMSCCPDPNPPIHKIVRKPSRILFLMD |
| | | GFDELQGAFDEHIGPLCTDWQKAERGDILLSSLIRKKLLPEASLLITTRPVALEK |
| | | LQHLLDHPRHVEILGFSEAKRKEYFFKYFSDEAQARAAFSLIQENEVLFTMCFI |
| | | PLVCWIVCTGLKQQMESGKSLAQTSKTTTAVYVFFLSSLLQPRGGSQEHGLC |
| | | AHLWGLCSLAADGIWNQKILFEESDLRNHGLQKADVSAFLRMNLFQKEVDCE |
| | | KFYSFIHMTFQEFFAAMYYLLEEEKEGRTNVPGSRLKLPSRDVTVLLENYGKF |
| | | EKGYLIFVVRFLFGLVNQERTSYLEKKLSCKISQQIRLELLKWIEVKAKAKKLQI |
| | | QPSQLELFYCLYEMQEEDFVQRAMDYFPKIEINLSTRMDHMVSSFCIENCHRV |
| | | ESLSLGFLHNMPKEEEEEEKEGRHLDMVQCVLPSSSHAACSHGLVNSHLTSS |
| | | FCRGLFSVLSTSQSLTELDLSDNSLGDPGMRVLCETLQHPGCNIRRLWLGRC |
| | | GLSHECCFDISLVLSSNQKLVELDLSDNALGDFGIRLLCVGLKHLLCNLKKLWL |
| | | VSCCLTSACCQDLASVLSTSHSLTRLYVGENALGDSGVAILCEKAKNPQCNLQ |
| | | KLGLVNSGLTSVCCSALSSVLSTNQNLTHLYLRGNTLGDKGIKLLCEGLLHPD |
| | | CKLQVLELDNCNLTSHCCWDLSTLLTSSQSLRKLSLGNNDLGDLGVMMFCEV |
| | | LKQQSCLLQNLGLSEMYFNYETKSALETLQEEKPELTVVFEPSW |
| SEQ ID | MBP- | MGKIEEGKLVIWINGDKGYNGLAEVGKKFEKDTGIKVTVEHPDKLEEKFPQVA |
| NO 3 | ΔNLRP3- | ATGDGPDIIFWAHDRFGGYAQSGLLAEITPDKAFQDKLYPFTWDAVRYNGKLI |
| | HIS | AYPIAVEALSLIYNKDLLPNPPKTWEEIPALDKELKAKGKSALMFNLQEPYFTW |
| | | PLIAADGGYAFKYENGKYDIKDVGVDNAGAKAGLTFLVDLIKNKHMNADTDYSI |
| | | AEAAFNKGETAMTINGPWAWSNIDTSKVNYGVTVLPTFKGQPSKPFVGVLSA |
| | | GINAASPNKELAKEFLENYLLTDEGLEAVNKDKPLGAVALKSYEEELAKDPRIA |
| | | ATMENAQKGEIMPNIPQMSAFWYAVRTAVINAASGRQTVDEALKDAQTGTDY |
| | | DIPTTENLYFQGYCAKYRAYVRSRFQCIEDRNARLGESVSLNKRYTRLRLIKEH |
| | | RSQQEREQELLAIGKTKTCESPVSPIKMELLFDPDDEHSEPVHTVVFQGAAGI |
| | | GKTILARKMMLDWASGTLYQDRFDYLFYIHCREVSLVTQRSLGDLIMSCCPDP |
| | | NPPIHKIVRKPSRILFLMDGFDELQGAFDEHIGPLCTDWQKAERGDILLSSLIRK |
| | | KLLPEASLLITTRPVALEKLQHLLDHPRHVEILGFSEAKRKEYFFKYFSDEAQAR |
| | | AAFSLIQENEVLFTMCFIPLVCWIVCTGLKQQMESGKSLAQTSKTTTAVYVFFL |
| | | SSLLQPRGGSQEHGLCAHLWGLCSLAADGIWNQKILFEESDLRNHGLQKADV |
| | | SAFLRMNLFQKEVDCEKFYSFIHMTFQEFFAAMYYLLEEEKEGRTNVPGSRLK |
| | | LPSRDVTVLLENYGKFEKGYLIFVVRFLFGLVNQERTSYLEKKLSCKISQQIRLE |
| | | LLKWIEVKAKAKKLQIQPSQLELFYCLYEMQEEDFVQRAMDYFPKIEINLSTRM |
| | | DHMVSSFCIENCHRVESLSLGFLHNMPKEEEEEEKEGRHLDMVQCVLPSSSH |
| | | AACSHGLVNSHLTSSFCRGLFSVLSTSQSLTELDLSDNSLGDPGMRVLCETLQ |
| | | HPGCNIRRLWLGRCGLSHECCFDISLVLSSNQKLVELDLSDNALGDFGIRLLC |
| | | VGLKHLLCNLKKLWLVSCCLTSACCQDLASVLSTSHSLTRLYVGENALGDSGV |
| | | AILCEKAKNPQCNLQKLGLVNSGLTSVCCSALSSVLSTNQNLTHLYLRGNTLG |
| | | DKGIKLLCEGLLHPDCKLQVLELDNCNLTSHCCWDLSTLLTSSQSLRKLSLGN |
| | | NDLGDLGVMMFCEVLKQQSCLLQNLGLSEMYFNYETKSALETLQEEKPELTV |
| | | VFEPSWHHHHHH |

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

To carry out the DEL-enabled discovery efforts with NLRP3, a combined screening library array of >500 billion molecules was used. To facilitate that discovery effort, specific choices in DEL library screening conditions enhanced the stability of NLRP3 with retention of ATPase activity during the DEL screen and employed several NLRP3 site blocking agents to enhance the ligand analysis efforts.

The following examples further illustrate aspects of the present disclosure. However, they are in no way a limitation of the teachings of the present disclosure as set forth.

### Example 1: Design and Optimization of Screening Construct

The DEL screen used a protein construct termed MBP-ANLRP3-HIS that contained the human NLRP3 protein sequence lacking the amino-terminal PYD domain. This protein also contained an amino-terminal maltose binding protein (MBP) domain and a carboxy-terminal HIS tag containing a tandem repeat of six histidine residues to facilitate purification of the protein and binding of the protein to nickel-charged MagBeads, respectively. MBP-ANLRP3-HIS was purified from Sf21(IPLB-Sf21-AE) insect cells and was produced in a 2-step purification process.

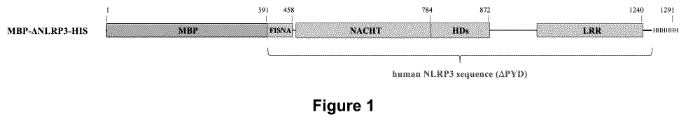

Peptide mass fingerprinting confirmed the protein identity while intact mass analysis confirmed the overall integrity of the protein and indicated the loss of the amino-terminal methionine. The theoretical molecular weight and isoelectric point (pI) of MBP-NLRP3-HIS are 145.30 kDa and 6.2, respectively. After one freeze-thaw cycle, the protein showed a highly pure band that eluted as a monodispersed sample in size exclusion chromatography (SEC). MBP-ANLRP3-HIS protein functionality was assessed based on ATPase activity using an ADP-Glo Kinase Assay (Promega). From these studies, it was determined that the Km of ATP to MBP-ANLRP3-HIS was 3.076 μM and that the MBP-ANLRP3-HIS protein showed sufficient activity in the ADP-GLO assay and DEL screen selection buffers. This protein construct was shown via its ATPase activity to have acceptable stability up to 2 hours in conditions mimicking one round of selection in the DEL screen. The full structure of MBP-ANLRP3-HIS is given in FIG. 1.

### Example 2: Setup of DEL Screening

### Setup of DEL Screening:

The DEL screen was organized to have the six separate screening trials identified in Table 1 with all DEL libraries screened in each trial. ATP was included in Trials 2-5 based on the findings that the presence of ATP resulted in exceptionally high NLRP3 binding for MCC950 (Coll, R. C., et al., Nat. Chem Biol., 2019, 15(6), 556-559). In addition, MCC950, CY-09 and tranilast were used as blocking agents in separate screening trials to establish an unbiased screening approach and to better understand the specific NLRP3 binding sites and mechanisms of action displayed by various ligands (Jiang, H., et al., J. Exp. Med., 2017, 214(11), 3219-3238), (Huang, Y., et al., EMBO Mol. Med, 2018, 10(4), 8689). As is standard screening practice, the MBP-ANLRP3-HIS protein construct was used in large molar excess over the anticipated fM concentration of individual screening compounds and the three blocking agents were each used at high uM levels to achieve maximum inhibitory effects.

TABLE 1
DEL screening paradigm with trials using blocking ligands.

| Trial | Target (250 pM) | Reagents |
|---|---|---|
| 1 | MBP-ΔNLRP3-HIS | None |
| 2 | MBP-ΔNLRP3-HIS | 1 mM ATP |
| 3 | MBP-ΔNLRP3-HIS | 1 mM ATP + 23.5 um CY-09 |
| 4 | MBP-ΔNLRP3-HIS | 1 mM ATP + 200 uM MCC950 |
| 5 | MBP-ΔNLRP3-HIS | 1 mM ATP + 200 uM tranilast |
| 6 | MBP-HIS | 1 mM ATP |

### Construction of the Screening Library:

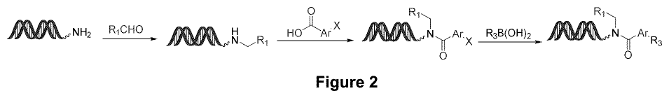

The DEL screen employed HitGen libraries of various constructions encompassing >500 billion compounds. The resulting screening data was analyzed to give both sequence count as the number of a particular DNA barcode that was observed in sequencing results and feature intensity as the calculated and normalized result of the enrichment of any given compound series. The sequence count arrays and distributions for binding were quite similar across Trials 2-5 suggesting that the binding of novel ligands to NLRP3 was not being altered significantly by any of the three blocking ligands. In addition, the most avid screening library ligands were neutral in charge in contrast to the anionic NLRP3 inhibitors MCC950, CY-09 and tranilast that were used as ligand blocking agents. The most interesting and relevant binding above background was displayed by compounds from a 98.8 million compound library generated via a 3-component synthetic plan that employed 521 aldehydes, 354 carboxylic acids and 528 borates as shown in FIG. 2.

### Example 3: Ligands Identified in DEL Screening

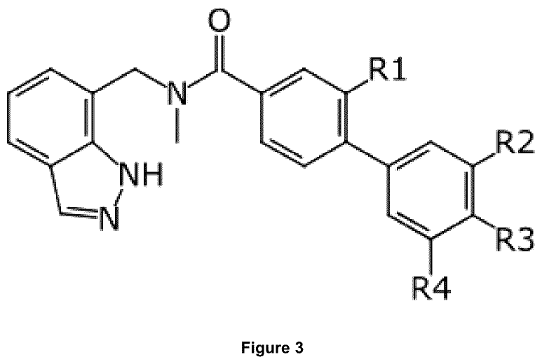

This library was found to provide a novel, high affinity group of neutral NLRP3 ligands that displayed an indazole scaffold functionalized at C-7 via a 3-atom carbonylaminomethyl linker connected to a biphenyl subunit as illustrated in generic structure 1 (FIG. 3). In general, a variety of substituents, i.e. R1, R2, R3 and R4 were present in the favored compounds that bound to NLRP3 and it was clear that the appropriate meta or para positioning of those substituents on the biphenyl unit was crucial for optimal binding. Specifically, the DEL-derived SAR for the compounds of highest affinity showed R1 to be an alkyl ether and R2, R3 and R4 were typically substituents selected from amino, cyano, trifluoromethyl, methylaminocarboxyl and fluoro. Of note, a variety of shorter and longer 3-atom linker sequences were incorporated into the library however the resulting compounds invariably lacked good binding potential.

### Example 4. Novel Indazole Class of Compounds

### Synthesis of Novel Indazole Class of Compounds:

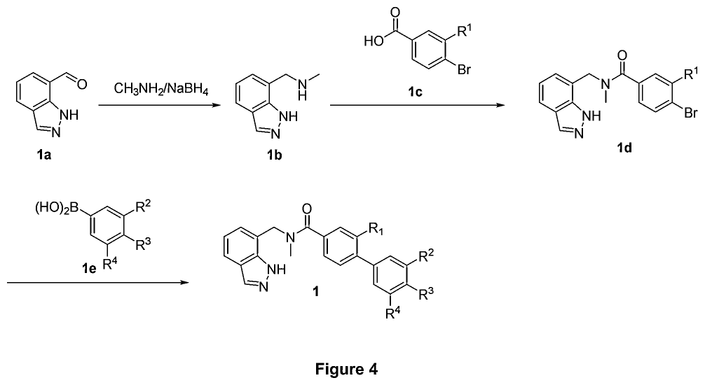

To follow up the initial DEL screen, examples of this novel indazole class of compounds were directly made ‘off-DNA’ and the functional activity of those resulting compounds determined by measuring their ability to inhibit IL-1P secretion in THP-1 cells. The synthesis of these novel indazole analogs was similar to the DEL plan and involved reductive amination of the 7-indazole carboxaldehyde 1a to provide the N-methyl derivative 1b (FIG. 4). That secondary amine was acylated with the appropriately derivatized acid 1c and the resulting amide 1d was converted to the biphenyl analog 1 using a functionalized boronic acid 1e in a Suzuki coupling.

### Test Potency of Compounds in Human Cells:

The potency of compounds for inhibition of NLRP3 activation was measured in the human monocytic THP-1 cell line which expresses high levels of NLRP3 inflammasome components and readily forms fully active NLRP3 inflammasome complexes under standard cell culturing conditions. Phorbol 12-myristate 13-acetate (PMA) pre-treatment of suspended THP-1 cells induces their adherence and partial differentiation into macrophage-like cells. Subsequent lipopolysaccharide (LPS) treatment acts as the ‘signal 1’ to induce a robust upregulation of pro-IL-1β while further addition of nigericin, a bacterial ionophore, acts as ‘signal 2’ to induce NLRP3 inflammasome formation and caspase-1 auto-cleavage to produce mature IL-1β. Under these conditions, compounds are screened for their ability to inhibit NLRP3 activation by measuring mature IL-1β levels by ELISA in cell culture supernatants (Zito, G. et al., Int. J. Mol. Sci., 2020, 21(12), 4294-4313).

Indazole compounds 2-8 (Table 2) were identified as high affinity binders from the DEL screen and were among the initial compounds prepared for evaluation in the THP-1 NLRP3 inflammasome activation assay. Rewardingly, the phenyl analog (2) along with the 3-fluoro-(3), 3-trifluoromethyl-(4), and 3-cynophenyl-(5) analogs all showed IC50 values <100 nM for inhibition of IL-1 secretion. Analogs 6, 7, and 8 were inactive at 20 uM in the THP-1 assay despite good binding profiles in the DEL screen. In addition, a variety of alternatively functionalized terminal phenyl ring analogs were prepared and the lack of activity of those compounds in the THP-1 assay closely mimicked the lack of efficient binding seen in the DEL screen.

TABLE 2
Indazole analog SAR at R2 position. Compound potency (IC50) was
determined by quantifying the decreases in IL-1β supernatant levels at
various compound concentrations using the THP-1 NLRP3
inflammasome activation assay.

1

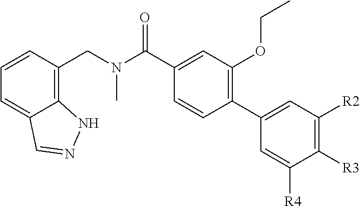

| Compound | R2 | R3 | R4 | IC50 (nM) | cLogD |
|---|---|---|---|---|---|
| 2 | H | H | H | 17 | 5.07 |
| 3 | F | H | H | 25 | 4.74 |
| 4 | CF3 | H | H | 52 | 5.35 |
| 5 | CN | H | H | 81 | 4.22 |
| 6 | NH2 | H | H | >20,000 | 3.92 |
| 7 | S(O)CH3 | H | H | >20,000 | 3.43 |
| 8 | C(O)NHCH3 | H | H | >20,000 | 3.63 |

The cell-active indazole-based inhibitors shown above were without precedent in the NLRP3 literature and their neutral charge character suggested good potential for both oral bioavailability and CNS permeability.

### Example 5. Buffer for Improved NLRP3 Variant Activity

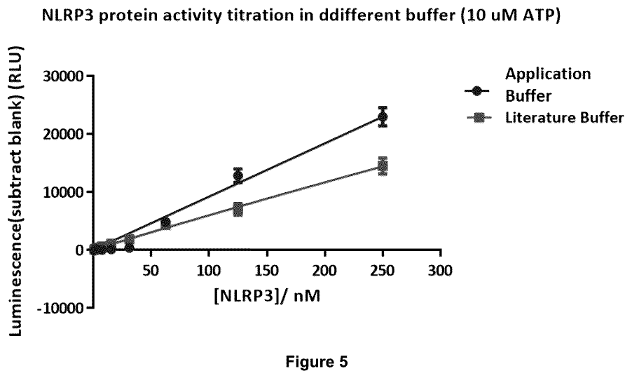

The DEL screen described above was performed with an optimized buffer to activity and stability of the NLRP3 variant. As shown in FIG. 5, the ATPase activity of the NLRP3 variant was measured in an ADP-Glo Activity Assay under the optimized buffer of the application and a literature buffer. Application buffer: 50 mM Tris-HCl, 150 mM NaCl, 10 mM MgCl2, 10% Glycerol, 0.005% Tween-20, pH=7.5. Literature buffer: 20 mM Tris-HCl, 133 mM NaCl, 20 mM MgCl2, 0.56 mM EDTA, 3 mM KCl, pH=7.8. Each buffer further comprised 10 μM ATP and a range of NLRP3 of 0 nM to 250 nM.

The data shows that the optimized buffer yielded an NLRP3 variant with increased ATPase activity.

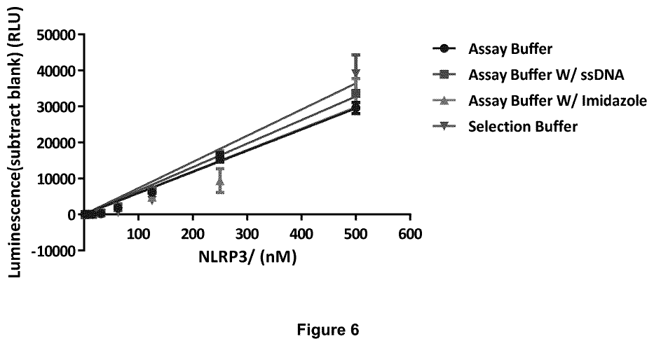

The inclusion of imidazole and ssDNA (which is a part of the DEL screen) was tested next. The assay buffer of FIG. 5 (Application buffer) was tested with 0.3 mg/mL ssDNA, 10 mM imidazole, or both. The tested buffers also included 3 μM ATP. The data shows that neither ssDNA or imidazole have a negative impact on the activity of the NLRP3 variant (FIG. 6).

1. A method of identifying a compound capable of binding to a Nucleotide-binding oligomerization domain, Leucine rich Repeat and Pyrin domain containing 3 (NLRP3) polypeptide, the method comprising:
1) contacting a compound suspected of being capable of binding an NLRP3 polypeptide with an NLRP3 variant, wherein the NLRP3 variant is immobilized on a surface;
2) washing the compound and immobilized NLRP3 variant with a buffer; and
3) detecting the compound,
wherein if the compound is detected, the compound is capable of binding an NLRP3 polypeptide,
wherein NLRP3 variant comprises at least a partial deletion of an amino terminal pyrin domain (PYD).

2. The method of claim 1, wherein the compound is linked to a polynucleotide sequence, optionally wherein detection of the compound comprises detecting the polynucleotide sequence, optionally wherein detecting the polynucleotide sequence comprises sequencing the polynucleotide sequence.

3-4. (canceled)

5. The method of claim 2, wherein;
the polynucleotide sequence comprises at least 5 nucleotides;
the polynucleotide sequence is between 5 and 50 nucleotides in length; and/or
the polynucleotide sequence is single stranded DNA (ssDNA).

6-7. (canceled)

8. The method of claim 1, wherein the NLRP3 variant is at a concentration of between 1 nM and 300 nM, optionally wherein the NLRP3 variant is at a concentration of between 25 nM and 250 nM.

9. (canceled)

10. The method of claim 1, wherein the NLRP3 variant is contained in a buffer comprising:
1) 5 mM to 500 mM Tris-HCl, optionally 50 mM Tris-HCl;
2) 15 to 1500 mM NaCl, optionally 150 mM NaCl;
3) 1 to 100 mM MgCl₂, optionally 10 mM MgCl₂;
4) 1% to 20% Glycerol, optionally 10% Glycerol; and
5) 0.0005% to 0.05% Tween-20, optionally 0.005% Tween-20,
optionally wherein the buffer further comprises:
1 to 500 mM imidazole, optionally 10 mM imidazole; and
0.03 to 3 mg/mL single-stranded DNA (ssDNA), optionally 0.3 mg/mL ssDNA.

11-13. (canceled)

14. The method of claim 1, wherein the contacting step 1) comprises incubating the compound with the NLRP3 variant and between 0.01 mM to 50 mM ATP, optionally between 0.1 mM to 1 mM ATP.

15. (canceled)

16. The method of claim 1, wherein the NLRP3 variant is immobilized on a solid support, optionally wherein the solid support comprises a nickel-immobilized resin.

17. (canceled)

18. The method of claim 1, further comprising:
4) incubating the compound capable of binding an NLRP3 polypeptide with a cell; and
5) measuring at least one pro-inflammatory cytokine secreted from the cell,
wherein a decrease in secretion of at least one pro-inflammatory cytokine from the cell indicates that the compound is an inhibitor of NLRP3, optionally wherein:
the at least one pro-inflammatory cytokine is IL-1β; and/or
the cell is a human monocytic THP-1 cell.

19-20. (canceled)

21. A method of identifying an inhibitor of Nucleotide-binding oligomerization domain, Leucine rich Repeat and Pyrin domain containing 3 (NLRP3), the method comprising:
1) contacting a compound suspected of being capable of binding an NLRP3 polypeptide with a NLRP3 variant, wherein the NLRP3 variant is immobilized on a surface;
2) washing the compound and immobilized NLRP3 variant with a buffer;
3) detecting the compound, wherein if the compound is detected, the compound is capable of binding an NLRP3 polypeptide;
4) incubating the compound capable of binding an NLRP3 polypeptide from step 3) with a cell; and
5) measuring at least one pro-inflammatory cytokine secreted from the cell,
wherein a decrease in secretion of at least one pro-inflammatory cytokine from the cell indicates that the compound is an inhibitor of NLRP3,
wherein NLRP3 variant comprises at least a partial deletion of an amino terminal pyrin domain (PYD), optionally wherein:
the at least one pro-inflammatory cytokine is IL-1β; and/or
the cell is a human monocytic THP-1 cell.

22-23. (canceled)

24. The method of claim 1, wherein the NLRP3 variant comprises a deletion of amino acids 1-142 of the amino acid sequence of SEQ ID NO: 1, optionally wherein the NLRP3 variant comprises an amino acid sequence with at least 80% identity to SEQ ID NO: 2.

25. (canceled)

26. The method of claim 1, wherein the NLRP3 variant is linked to one or more polypeptide domains, optionally wherein:
the one or more polypeptide domains comprise a solubility enhancing domain;
the one or more polypeptide domains are selected from the group consisting of maltose binding protein (MBP), glutathione S-transferase (GST), N-utilization substance A (NusA), and small ubiquitin-related modifier (SUMO); and/or
the one or more polypeptide domains comprise an affinity purification domain, optionally wherein the affinity purification domain comprises a His domain, 6×His domain, biotin, streptavidin, glutathione S-transferase (GST), FLAG (DYKDDDDK; SEQ ID NO: 4), and an antibody Fc domain, optionally wherein the NLRP3 variant comprises an amino acid sequence with at least 80% identity to SEQ ID NO: 3.

27-31. (canceled)

32. The method of claim 1, wherein:
the NLRP3 variant retains ATPase activity relative to a wild type NLRP3 of SEQ ID NO: 1;
the NLRP3 variant retains at least about 25% of ATPase activity relative to the ATPase activity of a wild type NLRP3 of SEQ ID NO: 1:
the NLRP3 variant retains the ability to form an NLRP3 inflammasome, relative to the ability of a wild type NLRP3 of SEQ ID NO: 1; and/or
the NLRP3 variant retains the ability to initiate release of IL-1p in a cell, relative to the ability of a wild type NLRP3 of SEQ ID NO: 1.

33-35. (canceled)

36. A method of treating an inflammatory disorder, comprising administering to a subject the NLRP3 inhibitor identified in claim 1.

37. A composition comprising:
1) an NLRP3 variant;
2) 5 mM to 500 mM Tris-HCl, optionally 50 mM Tris-HCl;
3) 15 to 1500 mM NaCl, optionally 150 mM NaCl;
4) 1 to 100 mM MgCl₂, optionally 10 mM MgCl₂;
5) 1% to 20% Glycerol, optionally 10% Glycerol; and
6) 0.0005% to 0.05% Tween-20, optionally 0.005% Tween-20,
wherein NLRP3 variant comprises at least a partial deletion of an amino terminal pyrin domain (PYD),
optionally wherein the composition further comprises:
1 to 500 mM imidazole, optionally 10 mM imidazole;
0.03 to 3 mg/mL single-stranded DNA (ssDNA), optionally 0.3 mg/mL ssDNA; and/or
between 0.01 mM to 50 mM ATP.

38-41. (canceled)

42. A Nucleotide-binding oligomerization domain, Leucine rich Repeat and Pyrin domain containing 3 (NLRP3) variant, comprising at least a partial deletion of an amino terminal pyrin domain (PYD).

43. The NLRP3 variant of claim 42, comprising a deletion of amino acids 1-142 of the amino acid sequence of SEQ ID NO: 1, optionally comprising an amino acid sequence with at least 80% identity to SEQ ID NO: 2, optionally wherein the NLRP3 variant is linked to one or more polypeptide domains, optionally wherein:
the one or more polypeptide domains comprise a solubility enhancing domain;
the one or more polypeptide domains are selected from the group consisting of maltose binding protein (MBP), glutathione S-transferase (GST), N-utilization substance A (NusA), and small ubiquitin-related modifier (SUMO); and/or
the one or more polypeptide domains comprise an affinity purification domain, optionally wherein the affinity purification domain comprises a His domain, 6×His domain, biotin, streptavidin, glutathione S-transferase (GST), FLAG (DYKDDDDK; SEQ ID NO: 4), and an antibody Fc domain, optionally wherein the NLRP3 variant comprises an amino acid sequence with at least 80% identity to SEQ ID NO: 3.

44-50. (canceled)

51. The NLRP3 variant of claim 42, wherein_:
the NLRP3 variant retains ATPase activity relative to a wild type NLRP3 of SEQ ID NO: 1;
the NLRP3 variant retains at least about 25% of ATPase activity relative to the ATPase activity of a wild type NLRP3 of SEQ ID NO: 1:
the NLRP3 variant retains the ability to form an NLRP3 inflammasome, relative to the ability of a wild type NLRP3 of SEQ ID NO: 1; and/or
the NLRP3 variant retains the ability to initiate release of IL-1p in a cell, relative to the ability of a wild type NLRP3 of SEQ ID NO: 1.

52-54. (canceled)

55. A nucleic acid encoding the NLRP3 variant of claim 42.

56. A vector comprising the nucleic acid of claim 55.

57. A host cell comprising the vector of claim 56, optionally wherein the host cell is an E. coli cell, a yeast cell, an insect cell, or a mammalian cell, optionally wherein the insect cell is an Sf21 cell.

58-59. (canceled)

60. A method of purifying the NLRP3 variant of claim 42, comprising:
1) introducing a vector encoding the NLRP3 variant into a host cell;
2) culturing the host cell under conditions to allow expression of the NLRP3 variant in the host cell; and
3) isolating the NLRP3 variant from the host cell, optionally wherein the isolating step 3) comprises contacting a lysate of the host cell with one or more affinity purification resins, optionally wherein the one or more affinity purification resins are selected from the group consisting of an immobilized metal-affinity chromatography (IMAC) resin, a maltose or amylose affinity chromatography resin, a glutathione affinity chromatography resin, a protein A affinity chromatography resin, and a anti-FLAG affinity chromatography resin.

61-62. (canceled)
